(19) United States
(12) Patent Application Publication
(10) Pub. No.: US 2020/0338221 A1
(43) Pub. Date: Oct. 29, 2020

(54) METHOD FOR IMPROVED LUMEN EXPOSURE IN DECONTAMINATION

(71) Applicant: Medivators Inc., Minneapolis, MN (US)

(72) Inventors: Judson A. Herrig, Elko New Market, MN (US); Lawrence John Formosa, Robbinsdale, MN (US); Maruti Sinha, Plymouth, MN (US); Huyen Bui, Brooklyn Park, MN (US)

(21) Appl. No.: 16/851,535

(22) Filed: Apr. 17, 2020

Related U.S. Application Data
(60) Provisional application No. 62/838,556, filed on Apr. 25, 2019.

(51) Int. Cl. A61L 2/18; A61L 2/22

(52) U.S. Cl. CPC A61L 2/186; A61L 2/18; A61L 2202/24; A61L 2202/15; A61L 2/22

(57) ABSTRACT

A method of reprocessing a lumen device is provided. The method includes the step of placing a lumen device with a proximal lumen end and a distal lumen end in a vacuum chamber. Liquid is purged from one or more channels of the lumen device by introducing a pressure differential at the first lumen end. Next, a decontamination fluid is delivered through the second lumen end of the lumen device.

### PRIORITY CLAIM

This application claims priority to and benefit of U.S. Provisional Application with Ser. No. 62/838,556 filed Apr. 25, 2019, entitled METHOD FOR IMPROVED LUMEN EXPOSURE IN DECONTAMINATION, which is herein incorporated by reference in its entirety.

### TECHNICAL FIELD

This disclosure relates generally to decontamination of medical devices; in particular, this disclosure relates to a method for preparing lumen(s) of a lumen device for decontamination to increase exposure of decontamination fluid during a decontamination process.

### BACKGROUND

Robust medical instruments are often sterilized at high temperatures. Commonly, the instruments are sterilized in a steam autoclave under a combination of high temperature and pressure. While such sterilization methods are very effective for more durable medical instruments, advanced medical instruments formed of rubber and plastic components with adhesives are delicate and wholly unsuited to the high temperatures and pressures associated with a conventional steam autoclave. Steam autoclaves have also been modified to operate under low pressure cycling programs to increase the rate of steam penetration into the medical devices or associated packages of medical devices undergoing sterilization. Steam sterilization using gravity, high pressure or pre-vacuum create an environment where rapid changes in temperature can take place. In particular, highly complex instruments which are often formed and assembled with very precise dimensions, close assembly tolerances, and sensitive optical components, such as endoscopes, may be destroyed or have their useful lives severely curtailed by harsh sterilization methods employing high temperatures and high or low pressures.

Endoscopes can also present problems in that such devices typically have numerous exterior crevices and interior lumens which can harbor microbes. Microbes can be found on surfaces in such crevices and interior lumens as well as on exterior surfaces of the endoscope. Other medical or dental instruments which comprise lumens, crevices, and the like can also provide challenges for decontaminating various internal and external surfaces that can harbor microbes.

In some cases, lumen devices may need decontamination while still in a wet condition. Unfortunately, the fluid or moisture within the lumen device can block decontamination fluid from completely decontaminating all internal surfaces. Existing decontamination systems typically require an extended drying time for wet lumen devices. This drying process increases the timing for decontaminating lumen devices and slows turnaround of decontaminated lumen devices to health care professionals.

Therefore, a need exists that overcomes one or more of the disadvantages of present decontamination systems.

### SUMMARY OF THE INVENTION

According to one aspect, this disclosure provides a method of reprocessing a lumen device. The method includes the step of placing a lumen device with a proximal lumen end and a distal lumen end in a vacuum chamber. Liquid is purged from one or more channels of the lumen device by introducing a pressure differential at the proximal lumen end. Next, a decontamination fluid is delivered to the distal lumen end of the lumen device.

According to another aspect, this disclosure provides a method of preparing an endoscope for reprocessing. The method includes the step of placing an endoscope with a first lumen end and a second lumen end in a vacuum chamber. The liquid is purged from one or more channels of the endoscope by introducing a vacuum at the first lumen end such that the purging step occurs prior to introducing any decontamination fluid into the endoscope.

### BRIEF DESCRIPTION OF THE DRAWINGS

The present disclosure will be described hereafter with reference to the attached drawings which are given as non-limiting examples only, in which:

FIG. 1 is diagrammatic view of a decontamination system according to an embodiment of the present disclosure; and

FIG. 2 is a flow chart showing example steps to prepare a lumen device for decontamination according to an embodiment of the present disclosure.

Corresponding reference characters indicate corresponding parts throughout the several views. The components in the figures are not necessarily to scale, emphasis instead being placed upon illustrating the principals of the invention. The exemplification set out herein illustrates embodiments of the invention, and such exemplification is not to be construed as limiting the scope of the invention in any manner.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the disclosure.

This disclosure relates to a decontamination system for decontaminating a lumen device. In some embodiments, the decontamination system includes a subsystem configured to prepare the lumen device for decontamination in a manner that purges liquid and/or moisture from internal channels, which increases exposure of decontamination fluid within the lumen device. Embodiments are also contemplated in which the purging subsystem for preparing the lumen device for decontamination could be a separate stand-alone system instead of being integrated into the decontamination system. For example, the lumen device could be placed in a decontamination preparation system with the purging subsystem to get the lumen device ready for decontamination, and then placed in the decontamination system to be decontaminated. Depending on the circumstances, a stand-alone purging system could also be used to dry out decontamination fluid from sterile devices. In some cases, this could be used in conjunction with high-level decontamination (HLD) and sterilization of medical instruments.

To prepare the lumen device for decontamination, the purging subsystem purges excess moisture or fluid that is blocking internal passageways in the lumen device prior to introducing decontamination fluid, which improves exposure to decontamination fluid. This also reduces the time needed to decontaminate (also called reprocess) the lumen device, and makes possible rapid decontamination of lumen devices with very small lumens, such as duodenoscopes with open elevator wire channels. Additionally, lumen devices may not be required to have a time-consuming drying period prior to entering the decontamination system. Moreover, this system could be used to rapidly dry lumen devices that are sterile, which is preferred by healthcare providers even if the lumen device is sterile.

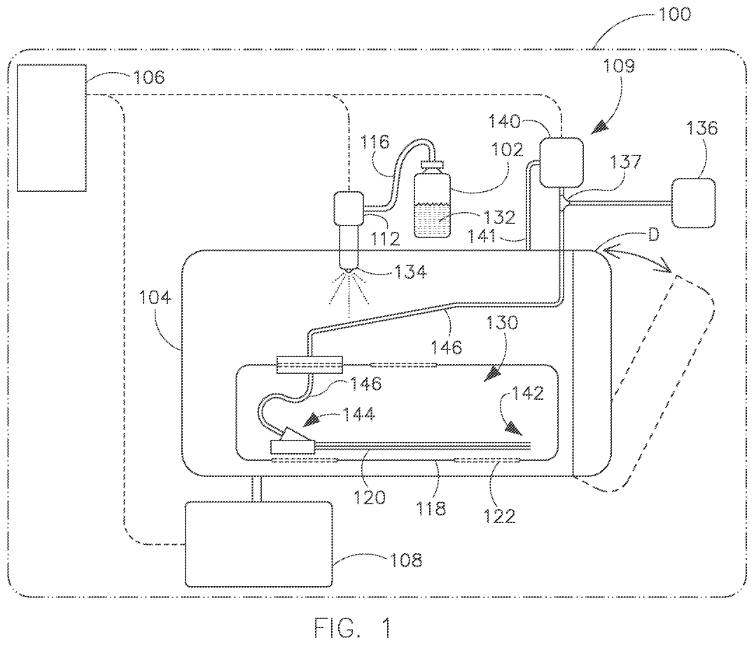

FIG. 1 is a diagrammatic view of one embodiment of a system 100 for decontaminating a medical, dental, or other device having one or more lumens extending there-through. Although FIG. 1 shows one connection for purposes of example, one skilled in the art should appreciate that multiple connections may be provided to connect with multiple lumens and ports on a lumen device. As discussed above, the system 100 includes a subsystem for preparing the lumen device for decontamination, which purges moisture and fluid from the lumen device 120, prior to introducing decontamination fluid. Although the example in FIG. 1 shows this subsystem integral with the decontamination system 100, as discussed above, this subsystem could be a stand-alone device that prepares the lumen device for decontamination prior to being placed in the decontamination system 100 or after decontamination.

As shown, the system 100 includes a reservoir 102, a decontamination chamber 104, a system controller 106, a chamber vacuum pump system 108, a purging system 109, and vaporizer 112 which is connected to the reservoir 102 by conduit 116. In some cases, helium gas can be used as a gas carrier for atomize decontamination fluid. Helium has a smaller density than air, and therefore, should move and diffuse the decontamination fluid better than air. This can be used to fill the decontamination chamber 104 an ideal pressure and can make good flow of decontamination fluid through channels in the lumen device 120.

In the example shown, a terminal package 118 containing a lumen device 120 for decontamination may be placed within the decontamination chamber 104. Depending on the circumstances, the terminal package 118 could be optional, such as embodiments in which the purging system is a stand-alone unit. In the illustrated embodiment, the terminal package 118 can include a plurality of openings or pores 122. The reservoir 102 may be in fluid communication with the decontamination chamber 104 via vaporizer 112 to supply decontamination fluid, which could be in vapor form, to the interior of the decontamination chamber 104. This decontamination fluid could be drawn into lumen device 120, such as via first end 142 of lumen device 120.

The system controller 106 provides control signals to and/or receives condition sensing and equipment status signals from the reservoir 102, the decontamination chamber 104, chamber vacuum pump system 108, purging system 109 and/or the vaporizers 110, 112. In some embodiments, the system 100 can be assembled in a device small enough to sit on a tabletop or counter. For example, the decontamination chamber 104 may have an interior volume of less than about ten cubic feet.

The lumen device 120 to be decontaminated can be placed into the decontamination chamber 104 by opening the door D and placing the lumen device 120 on a rack or other supporting assembly in the interior of the decontamination chamber 104. In some embodiments, the lumen device 120 may be enclosed in the terminal package 118 before being placed in the decontamination chamber 104. In the example shown, the terminal package 118 defines a lumen device receiving area 130 to receive the lumen device 120 for decontamination. In the illustrated embodiment, the terminal package 118 includes a plurality of openings or pores 122.

The reservoir 102 may be a holding tank or other assembly configured to hold a decontamination fluid 132. In some embodiments, the decontamination fluid 132 can be a chemical or other substance suitable for use in a sterilization process that complies with the International Organization for Standardization (ISO) standard ISO/TC 198, Sterilization of Healthcare Products and/or the Association for the Advancement of Medical Instrumentation (AAMI) standard ANSI/AAMI/ISO 11140-1:2005, “Sterilization of Healthcare Products—Chemical Indicators—Part I: General Requirements” (Arlington, Va.: AAMI 2005). In some embodiments, the decontamination fluid 132 can be a room temperature (e.g., 15° C. to 35° C.) substance that can be dispersed as a fluid, such as a liquid, a vapor, or a combination thereof (such as a fog) during the decontamination process. Suitable substances for the decontamination fluid 132 include hydrogen peroxide (H₂O₂) and peracetic acid (PAA). In various embodiments, the decontamination fluid is a composition that includes: (a) hydrogen peroxide; (b) organic acid; (c) a polymeric sulfonic acid resin based chelator; and (d) surfactant. The composition includes less than about 1 wt. % of an anticorrosive agent. The composition can further optionally include water.

In one aspect, the hydrogen peroxide present in the composition can be from about 0.5 wt. % to about 30 wt. %, from about 0.5 wt. % to about 1.5 wt. %, from about 0.8 wt. % to about 1.2 wt. %, from about 20 wt. % to about 30 wt. % and all ranges and values from about 0.5 wt. % to about 30 wt. %.

In another aspect, the acetic acid present in the composition can be from about 1 wt. % to about 25 wt. %, from about 4 wt. % to about 20 wt. %, from about 4.5 wt. % to about 5.5 wt. %, from about 9 wt. % to about 17 wt. % and all ranges and values from about 1 wt. % to about 25 wt. %.

In still another aspect, the peracetic acid present in the composition can be from about 0.01 wt. % to about 25 wt. %, from about 0.05 wt. % to about 20 wt. %, from about 0.05 wt. % to about 0.1 wt. %, from about 3.5 wt. % to about 8 wt. % and all ranges and values from about 0.01 wt. % to about 25 wt. %.

In yet another aspect, the polymeric resin chelator present in the composition can be from about 0.1 wt. % to about 5 wt. %, from about 0.2 wt. % to about 2 wt. %, from about 0.5 wt. % to about 1.5 wt. % and all ranges and value from about 0.1 wt. % to about 5 wt. %.

In various embodiments, the present invention provides for a composition that includes: (a) hydrogen peroxide, present in a concentration of about 0.5 wt. % to about 30 wt. %, e.g., about 28 wt. %; (b) acetic acid, present in a concentration of about 3 wt. % to about 25 wt. %, e.g., about 16 wt. %; (c) a sulfonic acid supported polymeric resin chelator present in a concentration of about 0.1 wt. % to about 5 wt. %, e.g., about 0.2 wt. % to about 0.7 wt. %; and, optionally, (d) Pluronic® 10R5 surfactant block copolymer, present in a concentration of about 2.0 wt. %, wherein the composition comprises less than about 0.1 wt. % of an anticorrosive agent, e.g., 0 wt. % of an anticorrosive agent. The composition can further optionally include water. In some embodiments, the hydrogen peroxide and acetic acid can combine to form peracetic acid, present in about 4 wt. % to about 8 wt. %, e.g., 6.8-7.5 wt. %.

In certain aspects, the peracetic acid/hydrogen peroxide compositions are stabilized without the need for a phosphonic based chelator, such as 1-hydroxyethylidene-1,1,-diphosphonic acid. In other aspects, a phosphonic based chelator, such as 1-hydroxyethylidene-1,1,-diphosphonic acid can be included in the sterilant fluid and therefore, component c), the polymeric sulfonic acid resin is optional.

The use of the polymeric stabilizer is detailed in pending PCT application PCT/US19/53090, filed Sep. 26, 2019, entitled “Peracetic Acid Stabilized Compositions with Polymeric Resins Chelators”, the contents of which are incorporated herein by reference.

The terminal package 118 is sized so that the lumen device 120 to be decontaminated fits within the terminal package 118. In some embodiments, the terminal package 118 may be generally described as having a top, a bottom, and four sides extending between the top and bottom to create a cube-like structure. However, the terminal package 118 may have any suitable shape which encloses the lumen device 120. In some embodiments, the terminal package 118 may be formed from a rigid material such that the terminal package 118 has a rigid or structured shape. Alternatively, the terminal package 118 may be formed from a flexible material such that the terminal package 118 has a flexible shape. Suitable materials for the terminal package 118 include but are not limited to a polymeric non-woven sheet, such as spun-bonded polyethylene (e.g., Tyvek®, sold by E.I. du Pont de Nemours and Company, Wilmington, Del.), and polymeric materials such as polyester and polypropylene. Suitable materials for the terminal package 118 having a rigid or structured shape include but are not limited to various metals such as aluminum, stainless steel and/or various polymers in rigid form such as polyethylene and/or polypropylene.

The lumen device 120 may be positioned within the terminal package 118 and subjected to one or more decontamination cycles. Suitable lumen devices include any medical, dental or other device having at least one lumen extending through at least a portion of the device. In some embodiments, the lumen device 120 may include at least one lumen extending the entire length of the device. For example, the lumen device 120 may be an endoscope.

The terminal package 118 may be configured to prevent or reduce microbes and/or other contaminants from entering the terminal package 118. In some embodiments, for example, the terminal package 118 can include a material suitable for allowing flow of a decontamination fluid (a sterilant), such as hydrogen peroxide (H₂O₂) and/or peracetic acid (PAA), into the lumen device receiving area 130 of the terminal package 118 and blocking or reducing the flow of contaminants into the interior of the terminal package 118. In the illustrated embodiment, the terminal package 118 includes a plurality of openings or pores 122 for allowing flow of the decontamination fluid 132 into the terminal package 118. In some embodiments, the pores 122 may be sized so as to allow the decontamination fluid 132 and/or air to communicate into and out of the container 118 as well as prevent microbes from entering the terminal package 118. In some embodiments, the terminal package 118 may have a direct fluid connection with fluid flow controlled through a valve instead of the plurality of openings 122.

In some embodiments, the decontamination fluid 132 can flow from the reservoir 102 to vaporizer 112 and subsequently to decontamination chamber 104 and lumen device 120. The decontamination fluid 132 from vaporizer 112 may decontaminate the internal surfaces of the lumen device. The amount of decontamination fluid 132 introduced into the decontamination chamber 104, the lumen device 120 or a combination thereof can be controlled by the system controller 106 by controlling the amount of the decontamination fluid 132 fed or delivered to vaporizer 112. The rate and amount of the decontamination fluid 132 delivered to vaporizer 112 may be preprogrammed into the system controller 106 or may be manually entered into the system controller 106 by a user of the system 100.

To decontaminate a lumen device 120, such as a medical, dental or other device, the lumen device 120 may be sealed within the terminal package 118 and placed in the decontamination chamber 104. The lumen device 120 is then subjected to a decontamination process which may include one or more decontamination cycles. In some embodiments, prior to subjecting the lumen device 120 to the decontamination cycles, the purging system 109 prepares the lumen device 120 for decontamination by purging moisture and fluid from the lumen device 120, which increases exposure for the application of decontamination fluid during the decontamination cycles. As discussed above, in some embodiments the purging system 109 could be a stand-alone system that purges the lumen device 120 before entering the decontamination chamber 104.

In some embodiments, the purging system 109 is configured to apply pressure and/or a vacuum source to the lumen device 120 to clear lumen channels so that liquid and/or moisture can be readily expelled. This purging increases contact of decontamination fluid with internal surfaces of lumen channels so there is sufficient contact between the decontamination fluid and all surfaces in the lumen device 120. There are numerous mechanisms that could be used to clear liquid and/or moisture from the lumen device's 120 channels prior to starting a decontamination process. The embodiment of purging system 109 shown in FIG. 1 is merely for purposes of illustration. Other embodiments are contemplated in which other mechanisms could be used to purge moisture and/or fluid from lumen device 120 prior to decontamination.

As shown, the purging system 109 includes a valve arrangement 136. Although the valve arrangement 136 is shown for purposes of illustration as a single valve, in some embodiments the valve arrangement could include multiple valves; in some cases, the valve arrangement could be embodied as a multiport valve to control fluid paths for purging the lumen device 120. In the embodiment shown, the purging system 109 includes a pump 140. Although this embodiment is shown as including the pump 140, one or more functions of the purging system could be performed by a reversible pump in the chamber vacuum pump system 108 depending on the circumstances.

In the embodiment shown, the lumen device 120 includes a first end 142 and a second end 144. As shown, the first end 142 is open to draw in decontamination fluid into the interior of the lumen device 120. As shown, the second end 144 of the lumen device 120 is connected to a conduit 146, which is in fluid communication with a tee-connection 137. A first end of the tee-connection 137 is fluidly connected to the valve arrangement 136 while the second end of the tee-connection 137 is fluidly connected to a pump 140. The valve arrangement 136 is movable between an open position that puts the second end 144 of the lumen device 120 in fluid communication with the atmosphere (out of the decontamination chamber 104) and a closed position that prevents flow out of the decontamination chamber 104 through the valve arrangement 136. The fluid connection with the pump 140 can be used to apply positive pressure to the second end 144 of the lumen device 120; in embodiments in which the pump 140 is reversible, the pump 140 could be used to apply positive or negative pressure to the second end 144 of the lumen device depending on the circumstances. In embodiments in which the pump 140 is a circulation pump, a conduit 141 could provide a fluid connection between the pump 140 and the decontamination chamber 104.

In some embodiments, the valve arrangement 136 is in a closed position during the pre-decontamination purging process so the second end 144 of the lumen device 120 is in fluid communication with the decontamination chamber 104. In some cases, the system 100 introduces a vacuum within the decontamination chamber 104, which helps to purge fluids and/or moisture from lumen device. By opening the valve arrangement 136, this allows fluid communication between the second end 144 of the lumen device 120 and the atmosphere. This pressure differential creates flow and clears fluid and/or moisture from the lumen(s) of the lumen device 120. In some embodiments, additional pressure could be applied above atmospheric pressure. For example, in some cases, a pressure of about 20 psi could be applied. In some embodiments, a source of compressed gas could be introduced at the first end 142 of the lumen device 120 to further purge lumen(s) of lumen device 120. Additionally, the pump 140 could apply positive and/or negative pressure to the second end 144 of the lumen device to further purge interior of lumen device 120. In some cases, the purging process may be replaced by an extended dry period. The purge step may be reduced or improved if done in a lower pressure environment such that the drying rate is increased. The closer the pressure in the lumen device 120 is to the decontamination fluid pressure, such as vapor pressure, the quicker that drying will occur.

After purging the lumen device 120 of moisture and fluid in preparation for decontamination, the lumen device 120 is subjected to one or more decontamination cycles. A suitable cycle may include adjusting the pressure of the decontamination chamber 104 to a suitable range, such as to a pressure less than 10 Torr, conditioning using plasma, and introducing the decontamination fluid 132 into the decontamination chamber 104 via vaporizer 112 and nozzle 134. The decontamination fluid 132 may be held within the decontamination chamber 104 for a period of time to facilitate the decontamination of the lumen device 120, and in particular, the exterior surfaces of the lumen device 120. Similarly, the decontamination fluid 132 may be held within the lumen device 120 for a period of time to facilitate the decontamination of the interior surfaces or lumen(s) of the lumen device 120. When the decontamination fluid 132 has been held in the decontamination chamber 104 for the desired or programmed amount of time, the system controller 106 can vent the decontamination chamber 104 to a higher, but sub-atmospheric pressure. The system controller 106 can then hold the pressure within the decontamination chamber 104 for a period of time to further facilitate the decontamination of the load. Following the hold period, the system controller 106 may evacuate the decontamination chamber 104 to remove the decontamination fluid residuals from the decontamination chamber 104 which may also include a plasma treatment to further enhance the removal of the substance residuals, followed by venting the decontamination chamber 104. This cycle or steps may be repeated or extended as part of a comprehensive cycle.

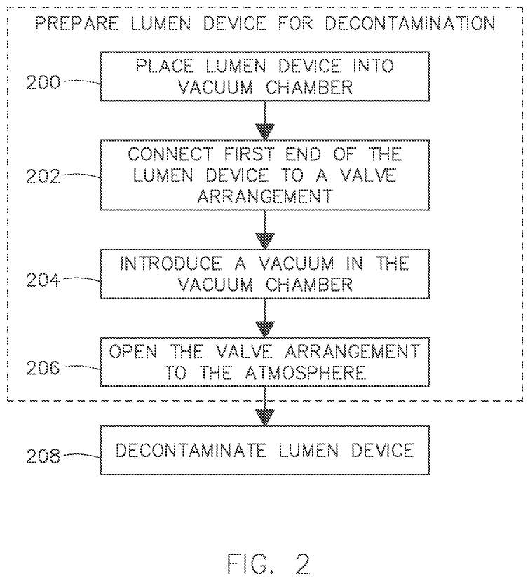

FIG. 2 is a flow chart showing example steps that may be taken to decontaminate the lumen device 120. In this embodiment, the lumen device 120 is first prepared for decontamination. For example, in block 200 the lumen device 120 may be placed in the decontamination chamber 104. The second end 144 of the lumen device is connected to a valve arrangement 136 (block 202). Next, the method steps to block 204 in which a vacuum is introduced in the decontamination chamber 204. The valve arrangement 136 is opened to the atmosphere to purge lumen(s) in lumen device 120 (block 206). The lumen device 120 is then decontaminated (block 208), which, as discussed above, may involve one or more decontamination cycles that involve adjusting the pressure of the decontamination chamber 104, conditioning using plasma, and introducing the decontamination fluid into the lumen device 120.

### EXAMPLES

Illustrative examples of the method and system disclosed herein are provided below. An embodiment of the method and system may include any one or more, and any combination of, the examples described below.

Example 1 is a method of reprocessing a lumen device. The method includes the step of placing a lumen device with a proximal lumen end and a distal lumen end in a vacuum chamber. Liquid is purged from one or more channels of the lumen device by introducing a pressure differential at the proximal lumen end. Next, a decontamination fluid is delivered to the distal lumen end of the lumen device.

In Example 2, the subject matter of Example 1 is further configured such that the purging step includes connecting the proximal lumen end to a valve arrangement that is in fluid communication to outside the vacuum chamber such that a valve arrangement is movable between an open position that allows fluid flow between the proximal lumen end and outside the chamber and a closed position that prevents fluid flow between the proximal lumen end and outside the vacuum chamber.

In Example 3, the subject matter of Example 2 is further configured such that the purging step includes introducing a vacuum into the vacuum chamber.

In Example 4, the subject matter of Example 3 is further configured such that the purging step includes opening the valve arrangement to the atmosphere.

In Example 5, the subject matter of Example 4 is further configured such that the purging step includes opening the valve arrangement to a source of compressed gas.

In Example 6, the subject matter of Example 5 is further configured such that the delivering step includes opening the valve arrangement and delivering a decontamination fluid to the proximal end of the lumen device.

In Example 7, the subject matter of Example 6 is further configured such that the delivering step includes drawing fluid from the proximal end of the lumen.

In Example 8, the subject matter of Example 2 is further configured such that the valve arrangement includes a plurality of valves.

In Example 9, the subject matter of Example 2 is further configured such that the valve arrangement is in fluid communication with a reversible pump.

In Example 10, the subject matter of Example 2 is further configured such that the valve arrangement comprises a multiport valve.

Example 11 is a method of preparing an endoscope for reprocessing. The method includes the step of placing an endoscope with a first lumen end and a second lumen end in a vacuum chamber. The liquid is purged from one or more channels of the endoscope by introducing a vacuum at the first lumen end such that the purging step occurs prior to introducing any decontamination fluid into the endoscope.

In Example 12, the subject matter of Example 11 is further configured such that the purging step includes connecting the first lumen end to a valve arrangement that is in fluid communication to outside the vacuum chamber such that a valve arrangement is movable between an open position that allows fluid flow between the first lumen end and outside the vacuum chamber and a closed position that prevents fluid flow between the first lumen end and outside the vacuum chamber.

In Example 13, the subject matter of Example 12 is further configured such that the purging step includes introducing a vacuum into the chamber.

In Example 14, the subject matter of Example 13 is further configured such that the purging step includes opening the valve arrangement to the atmosphere.

In Example 15, the subject matter of Example 14 is further configured such that the purging step includes opening the valve arrangement to a source of compressed gas.

In Example 16, the subject matter of Example 15 is further configured such that the delivering step includes opening the valve arrangement and delivering sterilant to the first lumen end of the endoscope.

In Example 17, the subject matter of Example 16 is further configured such that the delivering step includes drawing fluid from the first lumen end of the endo scope.

In Example 18, the subject matter of Example 12 is further configured such that the valve arrangement includes a plurality of valves.

In Example 19, the subject matter of Example 12 is further configured such that the valve arrangement is in fluid communication with a reversible pump.

In Example 20, the subject matter of Example 12 is further configured such that the valve arrangement comprises a multiport valve.

Although the present disclosure has been described with reference to particular means, materials and embodiments, from the foregoing description, one skilled in the art can easily ascertain the essential characteristics of the invention and various changes and modifications may be made to adapt the various uses and characteristics without departing from the spirit and scope of the invention.

What is claimed is:

1. A method of reprocessing a lumen device, the method comprising the steps of:
placing a lumen device with a first lumen end and a second lumen end in a vacuum chamber;
purging liquid from one or more channels of the lumen device by introducing a pressure differential at the first lumen end; and
delivering a decontamination fluid to the second lumen end of the lumen device.

2. The method of claim 1, where the purging step includes connecting the second lumen end to a valve arrangement that is in fluid communication to outside the vacuum chamber, wherein a valve arrangement is movable between an open position that allows fluid flow between the second lumen end and outside the chamber and a closed position that prevents fluid flow between the second lumen end and outside the vacuum chamber.

3. The method of claim 2, wherein the purging step includes introducing a vacuum into the vacuum chamber.

4. The method of claim 3, wherein the purging step includes opening the valve arrangement to the atmosphere.

5. The method of claim 4, wherein the purging step includes opening the valve arrangement to a source of compressed gas.

6. The method of claim 5, wherein the delivering step includes opening the valve arrangement and delivering a decontamination fluid to the second end of the lumen device.

7. The method of claim 6, wherein the delivering step includes drawing fluid from the first end of the lumen.

8. The method of claim 2, wherein the valve arrangement includes a plurality of valves.

9. The method of claim 2, wherein the valve arrangement is in fluid communication with a reversible pump.

10. The method of claim 2, wherein the valve arrangement comprises a multiport valve.

11. The method of claim 1, further comprising atomizing the decontamination fluid with a carrier gas comprising helium gas prior to delivering the decontamination fluid to the lumen device.

12. A method of preparing an endoscope for reprocessing, the method comprising the steps of:
placing an endoscope with a first lumen end and a second lumen end in a vacuum chamber; and
purging liquid from one or more channels of the endoscope by introducing a vacuum at the first lumen end, wherein the purging step occurs prior to introducing any decontamination fluid into the endoscope.

13. The method of claim 12, where the purging step includes connecting the second lumen end to a valve arrangement that is in fluid communication to outside the vacuum chamber, wherein a valve arrangement is movable between an open position that allows fluid flow between the second lumen end and outside the vacuum chamber and a closed position that prevents fluid flow between the second lumen end and outside the vacuum chamber.

14. The method of claim 13, wherein the purging step includes introducing a vacuum into the chamber.

15. The method of claim 14, wherein the purging step includes opening the valve arrangement to the atmosphere.

16. The method of claim 15, wherein the purging step includes opening the valve arrangement to a source of compressed gas.

17. The method of claim 16, wherein the delivering step includes opening the valve arrangement and delivering sterilant to the second lumen end of the endoscope.

18. The method of claim 17, wherein the delivering step includes drawing fluid from the first lumen end of the endoscope.

19. The method of claim 13, wherein the valve arrangement includes a plurality of valves.

20. The method of claim 13, wherein the valve arrangement is in fluid communication with a reversible pump.
